(19) United States
(12) Patent Application Publication
(10) Pub. No.: US 2023/0000945 A1
(43) Pub. Date: Jan. 5, 2023

(54) TOPICAL PHARMACEUTICAL FORMULATIONS OF A CYCLIC DEPSIPEPTIDE

(71) Applicant: Lifemax Laboratories, Inc., Redwood City, CA (US)

(72) Inventor: Laman Alani, Redwood City, CA (US)

(21) Appl. No.: 17/929,980

(22) Filed: Sep. 6, 2022

Related U.S. Application Data
(63) Continuation of application No. PCT/US2021/021138, filed on Mar. 5, 2021.
(60) Provisional application No. 62/986,526, filed on Mar. 6, 2020.

(51) Int. Cl. A61K 38/15; A61K 47/10; A61K 47/12; A61K 47/14; A61K 9/00; A61K 9/06; A61K 47/06; A61K 47/44; A61K 47/38

(52) U.S. Cl. CPC A61K 38/15; A61K 47/10; A61K 47/12; A61K 47/14; A61K 9/0014; A61K 9/06; A61K 47/06; A61K 47/44; A61K 47/38

(57) ABSTRACT

Disclosed here are pharmaceutical compositions of the compound of Formula (I) (LM030) suitable for topical administration. The compound can be in solubilized form, formulated with a solubilizing agent and one or more pharmaceutically acceptable excipients.

### CROSS REFERENCE TO RELATED APPLICATIONS

This application is a continuation of International Patent Application No. PCT/US2021/021138, filed Mar. 5, 2021, which claims the benefit under 35 U.S.C. § 119(e) of the U.S. Provisional Application Ser. No. 62/986,526 filed Mar. 6, 2020, the content of which is hereby incorporated by reference in its entirety.

### BACKGROUND

The cyclic depsipeptide of Formula (I)

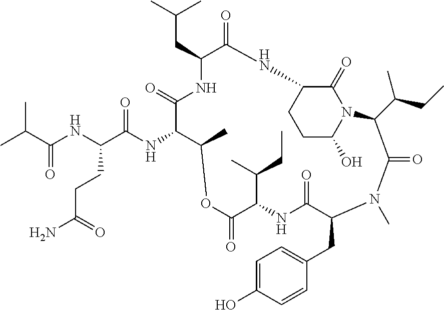 [SMILES: CC[C@H](C)[C@@H]1NC(=O)[C@H](Cc2ccc(O)cc2)N(C)C(=O)[C@H]([C@@H](C)CC)N2C(=O)[C@H](CC[C@H]2O)NC(=O)[C@H](CC(C)C)NC(=O)[C@@H](NC(=O)[C@H](CCC(N)=O)NC(=O)C(C)C)[C@@H](C)OC1=O]

is described in international patent application WO2009024527. It has been proposed for the treatment and prevention of certain skin disorders such as atopic dermatitis, psoriasis, pustular psoriasis, rosacea, keloids, hypertrophic scares, acne, Netherton's syndrome, and other pruritic dermatoses such as prurigo nodularis, unspecified itch of the elderly as well as other diseases with epithelial barrier dysfunction such as aged skin. However, its therapeutic applications have been limited by difficulties in formulating the compound into a formulation for topical administration with acceptable stability and adequate absorption through the skin using pharmaceutically acceptable excipients.

The compound of Formula (I), also referred to as LM030, is useful in the treatment of Netherton's Syndrome. Netherton Syndrome was first described by Comel in 1949 (Comel M, Dermatology 1949; 98: 133-136) and Netherton in 1958 (Netherton E W, Arch Dermatol. 1958; 78: 483-487). It is a severe autosomal recessive disease characterized by congenital erythroderma, “bamboo hair” and abnormality in the immune system (Bitoun E et al, Journal of Investigative Dermatology 2002; 118(2): 352-361). Netherton Syndrome in newborns can be life-threatening due to missing skin protection that leads to severe dehydration, hypernatremia, hypothermia, gross weight loss and sepsis. Failure to thrive is common in childhood as a result of chronic erythroderma, persistent cutaneous infection, malnutrition and metabolic disorders (Jones S K et al, Br. J. Dermatol. 1986; 114: 741-743; Judge M R et al, Br. J. Dermatol. 1994; 131: 615-621). The severity of the skin abnormality in older patients can fluctuate over time. Most Netherton Syndrome patients are also inflicted with immune system-related disorders such as food allergies and asthma.

Netherton Syndrome is caused by mutations in the SPINK5 gene that encodes a serine peptidase inhibitor, Lympho-epithelial Kazal-type-related inhibitor (LEKTI)Chavanas et al 2000; Nat. Genet. 25:141-142.) The loss of LEKTI leads to dysregulation of epidermal proteases and severe skin barrier impairment. Kallikrien-related peptidases, which are inhibited by LEKTI, are reported to play major roles in Netherton Syndrome pathology (Kasparek P et al, PLOS Genetics 2017, 13(1); Caubet C et al, Journal of Investigative Dermatology 2004; 122: 1235-1244).

Atopic dermatitis is also known as atopic eczema and is an inflammatory skin disease. It causes the skin to become inflamed and irritated, making it extremely itchy. Scratching may cause redness, swelling and cracking. While the condition may occur at any age, it typically starts in childhood and may change in severity over time.

Improved topical formulations of LM030 are needed for the treatment of Netherton's Syndrome, atopic dermatitis, and other skin diseases and disorders.

### SUMMARY

The present invention provides a pharmaceutical composition of the compound of Formula (I) (LM030), wherein LM030 is in solubilized form with a solubilizing agent and one or more pharmaceutically acceptable excipients, in a formulation suitable for topical administration.

It has now been found that LM030 in solubilized form with a solubilizing agent may be incorporated into topical pharmaceutical formulations as described herein, wherein the resulting topical formulations maintain good stability while also achieving high bioavailability of LM030 in the skin with low systemic exposure and much lower variability than previous formulations. In a preferred topical formulation, the solubilized LM030 and solubilizing agent can be incorporated into a matrix, typically a hydrophobic matrix.

In some embodiments, the solubilizing agent is selected from the group consisting of diethylene glycol monoethyl ether, medium chain triglycerides, fatty acids, propylene glycol and combinations thereof. In some preferred embodiments, the solubilizing agent is diethylene glycol monoethyl ether, alone or combination with a fatty acid such as oleic acid. In some embodiments, the solubilizing agent is a PEG-fatty acid derivative. In some embodiments, the solubilizing agent is a medium-chain triglyceride or a mixture of medium-chain triglycerides.

In some embodiments, the hydrophobic matrix comprises one or more excipients selected from the group consisting of paraffins, vegetable oils, animal fats, synthetic glycerides, waxes, perfluorocarbons, semiperfluorocarbons, liquid polysiloxanes and combinations thereof. In some embodiments, the hydrophobic matrix comprises one or more excipients selected from the group consisting of petrolatum, mineral oil and isopropyl myristate and combinations thereof.

The formulations of the present invention may further comprise a surfactant and/or a consistency enhancer. In a preferred embodiment, the formulation contains microcrystalline wax as a consistency enhancer. Formulations of the invention are suitable for topical administration and may be prepared as a gel, cream, ointment, lotion, spray or foam.

In some embodiments, the active agent is present in the formulation in an amount of from about 0.1 to about 5% w/w of the composition. In some embodiments, the solubilizing agent is present in the formulation in an amount of from about 2.5% to about 25% (w/w) of the composition. When diethylene glycol monoethyl ether is the solubilizing agent, it is preferably in an amount of from about 2.5% (w/w) to about 10% (w/w) of the composition.

In a preferred embodiment, the formulation is an ointment and comprises diethylene glycol monoethyl ether as the solubilizing agent, microcrystalline wax as a consistency enhancer and a hydrophobic matrix of petrolatum, mineral oil and isopropyl myristate. The formulations of the invention may be used to treat Netherton's disease, atopic dermatitis and other skin diseases and disorders.

Also provided are methods of treating Netherton's Disease comprising topical administration of the pharmaceutical compositions described herein to a patient in need thereof. The topical pharmaceutical compositions will comprise an effective amount of LM030, preferably in an amount of from about 0.1% to about 5% (w/w), more preferably in an amount of from about 0.2 to about 1% (w/w), and most preferably in an amount of about 1% (w/w). The compositions may be applied on affected area on the body either once a day or, preferably, twice a day or on as needed bases more than twice a day. The composition may be used chronically or on an as-needed bases.

### DETAILED DESCRIPTION

The compound of Formula I, also referred to herein as LM030, presents significant challenges in the preparation of a suitable topical formulation. Suitable topical formulations require permeation and penetration properties sufficient to deliver therapeutic quantities of the active agent to the affected layer in the skin, while preferably minimizing systemic exposure in order to minimize systemic side effects. At the same time, for practical use, the formulation must exhibit adequate storage stability to support acceptable shelf life for commercialization purposes.

LM030 shows only moderate solubility in water and aqueous buffers, and low solubility in lipophilic excipients. In polar organic solvents, it demonstrates good solubility, but stability is poor. In addition, when the compound is not solubilized, such as when provided in a suspension formulation, it shows poor absorption leading to sub-therapeutic drug levels in the target layer of the skin and very high variability, leading to unpredictable treatment effects. Difficulties in formulating LM030 for topical administration are described in U.S. Pat. No. 8,680,054. In particular, the solubility and stability properties of LM030 create challenges in developing a topical formulation with adequate storage stability that is also able to provide an effective amount of active agent available for skin penetration [See col 4, lines 16-23].

The formulations of the present invention show good storage stability, which is comparable to the stability observed by the suspension formulations described in U.S. Pat. No. 8,680,054, while also achieving high absorption of LM030 in the skin with low systemic exposure and much lower variability.

As used in the present specification, the following words, phrases and symbols are generally intended to have the meanings as set forth below, except to the extent that the context in which they are used indicates otherwise.

Reference to the compound of Formula I, or LM030, or “active agent,” as used in the formulations of the present invention, includes the compound in amorphous and crystalline forms, such as polymorphs, as well as analogs, solvates, prodrugs, complexes and pharmaceutically acceptable salts thereof. Also included are various sizes, or milled forms, of the compound, such as micronized particles.

“Pharmaceutically acceptable salts” refers to the nontoxic acid or alkaline earth metal salts of the compound of Formula I that are acceptable for use in topical pharmaceutical preparations. Representative salts include: acetate, adipate, alginate, citrate, aspartate, benzoate, benzene-sulfonate, bisulfate, butyrate, camphorate, camphorsulfonate, digluconate, cyclopentanepopionate, dodecylsulfate, ethanesulfonate, glucoheptanoate, glycerophphosphate, hemisulfate, heptanoate, hexanoate, fumarate, hydrochloride, hydrobromide, hydroiodide, 2-hydroxyethanesulfonate, lactate, maleate, methane-sulfonate, nicotinate, 2-naphth-alenesulfonate, oxalate, pamoate, pectinate, persulfate, 3-phenyl-propionate, picrate, pivalate, propionate, succinate, sulfate, tartrate, thiocyanate, p-toluene-sulfonate, and undecanoate. Also, basic nitrogen-containing groups can be quarternized with such agents as alkyl. Halides, such as methyl, ethyl, propyl, and butyl chloride, bromides, and iodides; dialkyl sulfates like dimethyl, diethyl, dibutyl, and diamyl sulfates, long chain halides such as decyl lauryl, myristyl, and stearyl chlorides, bromides and iodides, aralkyl halides like benzyl and phenethyl bromides, and others. Basic addition salts can be prepared in situ during the final isolation and purification of the compounds, or by separately reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include cations based on the alkali and alkaline earth metals, such as sodium, lithium, potassium, calcium, magnesium, aluminum salts and the like, as well as nontoxic ammonium, quaternary ammonium, and amine cations, including ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine, and ornithine.

“Topical administration” and compositions suitable for “topical administration,” as used herein has the meaning known in the field. See, e.g., European Pharmacopoeia, 6.3, 01/2009, 0132. Pharmaceutical compositions suitable for topical administration typically include liquid and semi-solid forms, adapted for topical application. Such forms include liquid solutions and suspensions, tinctures, gels, patches, foams, ointments, lotions, sticks or sprays.

As used herein, “treat,” “treatment” or “treating” of a disease or disorder refers to ameliorating the disease or disorder, such as by slowing or arresting the progression of the disease or disorder, or by alleviating at least one symptom thereof. “Treat,” “treatment” or “treating” may also refer to the prevention or delaying the onset or development of the disease or disorder.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the singular forms “a” and “the” include plural references unless the context clearly dictates otherwise.

The pharmaceutical compositions of the present invention comprise the active agent in solubilized form with a solubilizing agent with one or more pharmaceutically acceptable excipients, in a final formulation suitable for topical administration. The aolubilized active agent and solubilizing agent will be within a matrix, preferably a hydrophobic matrix.

The compound of Formula I may be obtained by the methods described in WO2009024527. The amount of active agent in the compositions of the invention can vary over a range, with the effective amount dependent of the condition to be treated. Typically, the active agent will be in an amount of about 0.1% to about 5% (w/w), preferably from about 0.2% to about 2.0% (w/w), and most preferably about 0.5% to about 1.0% (w/w).

Formulations containing LM030 in solubilized form with a solubilizing agent within a matrix, as described herein, surprisingly show significantly improved stability over any other solubilized formulations and comparable stability as compared to suspension formulations of the prior art formulations. Further, the formulations of the invention show significantly higher bioavailability of the active agent in the desired layer of the skin, and low systemic exposure with much lower variability than the prior art formulation. The formulations of the present invention may also be used with other peptides having similar formulation challenges for topical administration, including other peptide inhibitors of kallikrein 5 or kallikrein 7. Such peptides may be of from about 4 to about 30 amino acids, including, e.g., filaggrin.

Solubilizing agents are known in the art and may be selected by the skilled artisan to be compatible with the final formulation. See, De Villiers, Melgardt (2009) Pharmaceutical Solvents and Solubilizing Agents. In: Judith E. Thompson (ed), A Practical Guide to Contemporary Pharmacy Practice, Ed. 3, Chapter 15, Lippincott, Williams and Wilkins.

Preferred solubilizing agents are pharmaceutically acceptable excipient that help in solubilizing the active agent without compromising stability and are compatible with the topical delivery system. Also, it is non-irritating and suitable for chronic application, such that it may be used on a compromised skin such as that which occurs in Atopic Dermatitis (AD) and Netherton Syndrome (NS). Suitable solubilizing agents include glycols, such as glycerin, propylene glycol, and polyethylene glycol; glycol ethers such as diethylene glycol monoethyl ether (sold as Transcutol®), (also called di(ethylene glycol) ethyl ether, (2-(2-ethoxyethoxy) ethanol, ethyldiglycol, diEGEE); medium chain triglycerides (MCT) (e.g, 6-12 carbons in length) and mixtures of MCTs, such as coconut oil and Miglyol 812 (See Buss, N., et al., J Appl Toxicol. (2018) (38(10):1293-1301); and fatty acids such as oleic acid and linoleic acid. In some embodiments, the solubilizing agent is a PEG-fatty acid derivative, (Casiraghi A., Selmin F., Minghetti P., Cilurzo F., Montanari L. (2015) Nonionic Surfactants: Polyethylene Glycol (PEG) Ethers and Fatty Acid Esters as Penetration Enhancers. In: Dragicevic N., Maibach H. (eds) Percutaneous Penetration Enhancers Chemical Methods in Penetration Enhancement. Springer, Berlin, Heidelberg), such as Labrasol®, a PEG derivative of medium chain fatty acid triglyceride of capric and caprylic acid. See Negi, J. S., (2019) Nanolipid Materials for Drug Delivery Systems, in: Mohapatra, S. S., Ranjan, S., Dasgupta, N., Mishra R. K., and Thomas, S. (eds), Characterization and Biology of Nanomaterials for Drug Delivery, 2019. Further, the solubilizing agent may be Labrafac™ lipophils WL 1349, supplied by Gattefosse, S. A. (https://www.cphi-online.com/labrafactm-lipophile-w1-1349-prod486142.html).

The amount of the solubilizing agent in the formulation will typically be about 1.0% (w/w) to 50% (w/w) of the composition, and preferably between 2.5% and 15% (w/w) of the composition, and most preferably between 5% and 10% (w/w) of the composition. In some embodiments, the solubilizing agent is diethylene glycol monoethyl ether, and is present in an amount of about 2.5% to about 10% (w/w) of the composition, preferably, from about 5% to about 10% (w/w) of the composition. In further embodiments, the solubilizing agent is a mixture of hydrophobic and hydrophilic solvents, such as a mixture of diethylene glycol monoethyl ether and oleic acid in various ratios, or mixtures including polyethylene glycol or PEG in combinations with diethylene glycol monoethyl ether and/or oleic acid in various ratios. For example, diethylene glycol monoethyl ether and oleic acid may each be present in an amount of about 2.5% (w/w) of the composition.

In some embodiments, a hydrophobic matrix provides a matrix for the solubilized active agent. The hydrophobic matrix excipients will be selected to incorporate an effective amount of solubilized LM030, without compromising the stability of the final formulation and will provide the desired viscosity of the composition for ease of application by the end user. Pharmaceutically acceptable excipients for use in the hydrophobic matrix are known in the art, and include paraffins, vegetable oils, animal fats, synthetic glycerides, waxes, perfluorocarbons, semiperfluorocarbons and/or liquid polysiloxanes, and mixtures thereof. Suitable materials further include solid and liquid hydrocarbons, which may be linear or branched. Preferred hydrophobic materials include mineral oil, petrolatum and microcrystalline wax. In some embodiments, the composition comprises a mixture of mineral oil, petrolatum and microcrystalline wax

The hydrophobic matrix components may make up from about 50% to about 95% (w/w) of the final composition. In some embodiments, the hydrophobic matrix comprises up to about 65% (w/w) mineral oil. In some embodiments, the composition comprises about 20% to about 40% (w/w) mineral oil. The composition may contain up to about 95% (w/w) petrolatum. In embodiments where the final formulation is an ointment, the composition may contain from about 60% to about 80% (w/w) of petrolatum, and most preferably contains from about 65% to about 70% (w/w) petrolatum. The composition may contain up to about 25% (w/w) microcrystalline wax, and preferably of from about 0% to about 10% (w/w) microcrystalline wax. In some embodiments, the hydrophobic matrix contains a mixture of petrolatum and mineral oil in a ratio of from about 1:1 to about 10:1, preferably of from about 2:1 to about 4:1.

In some embodiments, the compositions of the invention further comprise a consistency enhancer. Consistency enhancers are known in the art. Suitable consistency enhancers include saturated fatty acids and saturated fatty acid esters. In a preferred embodiment, the isopropyl myristate is incorporated as a consistency enhancer.

Additional excipients useful in the preparation of topical pharmaceutical formulations may be additionally incorporated into the formulations of the present invention. Excipients for topical pharmaceutical formulations are known in the art and may be selected based on desired properties of the final formulation. See, e.g., Handbook of Pharmaceutical Excipients, Rowe, R. C. and Shesksy, P. J., et al., (2012). Components such as surfactants, stiffening or thickening agents, emollient, penetration enhancer, preservative, anti-microbial agents, and the like. For example, the final formulation will be adapted to be delivered in the preferred dosage form, e.g., a lotion, cream, ointment, spray or foam, and will be optimized to minimize skin irritation.

Surfactants may be utilized advantageously to increase percutaneous absorption, penetration enhancement or release rate. Examples of such surfactants include, but are not limited to ceteareth-20 available as CETOMACROGOL.® 1000, glycerol monostearate, glycerol distearate, glyceryl stearate, polyoxyethylene stearate, a blend of glyceryl stearate and PEG-100 stearate, (As ArLACEL 1 65), polysorbate 40, polysorbate 60, polysorbate 80, CETETH-20.™, sorbitan monopalmitate, sorbitan monostearate, sorbitan monooleate, sorbitan sesquioleate, and mixtures thereof. The amount of surfactant employed will generally be from about 0.5% (w/w) to about 10% (w/w).

Penetration enhancers may be incorporated in the present invention and will be selected to maintain stability of the active agent. Penetration enhancers useful in the formulations of the present invention include, for example, borage oil, eucalyptus oil (e.g., eucalyptus globulus oil, Eucalyptus tereticortis oil, tetrahydropiperine (THP), fatty alcohols (e.g., myristyl alcohol, cetyl alcohol, stearyl alcohol), fatty acids (e.g., oleic acid), fatty acid esters (e.g., isopropyl myristate, isopropyl palmitate), polyols (e.g., propylene glycol, polyethylene glycol, glycerol), polyethylene glycol monolaurate, lecithin, poloxamers, Labrofac® lipophile WL 1349, Miglyol® triglycerides), and the like. Other suitable penetration enhancers include, but are not limited to, diethylene glycol, n-decyl methyl sulfoxide, dimethyl sulfoxide, dimethylacetamide, laurocapram, dimethylformamide, sucrose monooleate, amides and other nitrogenous compounds (e.g., urea, 2-pyrrolidone, 1-methyl-2-pyrrolidone, ethanolamine, diethanolamine and triethanolamine), terpenes, alkanones, organic acids (e.g., citric acid and succinic acid) and N-methyl-2-pyrrolidine (Pharmasolve®), or combinations thereof.

The formulations may also contain oxidation mitigating additives to minimize or eliminate the possibility of skin irritation or skin damage. Suitable irritation-mitigating additives include, for example: a-tocopherol; monoamine oxidase inhibitors, particularly phenyl alcohols such as 2-phenyl-1-ethanol; glycerin; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing from about 0.05% to about 1.0% (w/w).

Antioxidants may be included as known in the art. Suitable antioxidants include phenol derivatives (e.g., butylated hydroxytoluene (BHT), butylated hydroxyanisole (BHA)), ascorbic acid derivatives (ascorbic acid, ascorbyl palmitate), tocopherol derivatives (e.g., Vitamin E, vitamin E TPGS), bisulfite derivatives (Na bisulfite, Na meta bisulfite), thio urea and combinations thereof. An antioxidant will typically be in an amount of from about 0.005% to about 0.5% (w/w).

Preservatives are known in the art and may be included to increase the shelf-life of the compositions. Suitable preservatives include phenols, and parahydroxybenzoates, parabens, biguanides, mercuric salts, and imidurea. Preservatives may be present in amounts of from about 0.01% to about 3% (w/w).

In some embodiments, the pharmaceutical formulations of the present disclosure do not include a substantial amount of a LM030 suspension. In one embodiment, less than about 15%, 10%, 5%, 2%, 1%, 0.5%, or 0.1% of LM030 of the pharmaceutical formulation is in a suspension condition.

The pharmaceutical formulations of the invention may be incorporated into any suitable topical dosage form, including, for example, ointments, creams, lotions, gels, foams and sprays.

Ointments, as is well known in the art, are semisolid preparations that are typically based on petrolatum or petroleum derivatives. As with other carriers or vehicles, an ointment base is typically inert, stable, non-irritating and non-sensitizing. Ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (w/o) emulsions or oil-in-water (o/w) emulsions, and include, for example, cetyl alcohol, glyceryl monostearate, lanolin, stearic acid and polyethylene glycols of varying molecular weight, or combinations thereof. Other examples of suitable hydrocarbon bases include, but are not limited to, hard, soft, or liquid paraffin, glycerol, beeswax, a metallic soap, a mucilage, an oil of natural origin (such as almond, corn, arachis, castor or olive oil), wool fat or its derivative, a fatty acid (such as stearic acid or oleic acid), or combinations thereof.

Creams, as also well known in the art, are viscous liquid or semisolid oil-in-water emulsions. Cream bases can be water-washable, and contain an oil phase, an emulsifier, and an aqueous phase. The oil phase, also called the “internal” phase, may include petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant, or combinations thereof.

Gel formulations are typically semisolid, suspension-type systems. Single-phase gels typically contain organic macromolecules distributed substantially uniformly throughout the carrier liquid, which is typically aqueous, but may also contain an oil. Exemplary “organic macromolecules” (i.e., gelling agents), are crosslinked acrylic acid polymers such as the “carbomer” family of polymers (e.g., carboxypolyalkylenes that may be obtained commercially as Carbopol®). Also exemplified are hydrophilic polymers such as polyethylene oxides, polyoxyethylene-polyoxypropylene copolymers, polyvinyl alcohol and polyvinyl pyrrolidones; cellulosic polymers such as hydroxypropyl cellulose, hydroxyethyl cellulose, hydroxypropyl methylcellulose, carboxymethyl cellulose, hydroxypropyl methylcellulose phthalate, and methyl cellulose; gums such as carrageenan gum, tragacanth and xanthan gum; sodium alginate; and gelatin. Other examples of suitable gel-forming agents include carboxypoly-methylene derivatives such as Carbopol and pectins.

Lotions are typically preparations to be applied to the skin surface without friction, and are typically liquid or semi-solid preparations. Lotions are usually suspensions of solids, and may comprise a liquid oily emulsion of the oil-in-water type. Lotions can be used to treat large body areas because of the ease of applying a more fluid composition. Thus, lotions will typically contain compounds useful for localizing and holding the active agent in contact with the skin (e.g., methylcellulose), sodium carboxymethyl-cellulose, or the like. They may further include a moisturizer, such as glycerol, or an oil, such as castor oil or arachis oil.

Pharmaceutical foam formulations are known in the art. A foam preferably includes at least one foaming agent such as a protein or surfactant. Surfactants stabilize the foam, e.g., by inhibiting bubble coalescence. See, Zhao, Y.; Brown, M. B.; Jones, S. J., Pharmaceutical foams: are they the answer to the dilemma of topical nanoparticles? Nanomedicine (2010). Qualities such as foam stability, easiness to spread, and appropriate breakability upon application to the skin are features that may be optimized. These characteristics can be measured by conducting foam formation and foam collapsibility experiments. Foam formation (foam height vs. time), for example, is predictive of the generation of a sprayable/spreadable foam.

Many foams are generated by dispensing a foam base in combination with a dissolved, gaseous propellant that expands upon release from a container to produce the foam's bubbles (e.g., those disclosed in WO 2010/125470).

In certain preferred embodiments the topical formulation of the present invention is an ointment. In these preferred embodiments, the LM030 is present in the formulation in an amount of from about 0.1 to about 5% (w/w) of the composition, preferably at about 0.5 to about 1% (w/w). Preferably, the solubilizing agent is present in the formulation in an amount of from about 1.0% to about 25% (w/w) of the composition. When diethylene glycol monoethyl ether is the solubilizing agent, it is preferably of from about 2.5% (w/w) to about 15% (w/w) of the composition, most preferably of from about 5% to about 10% (w/w). In a preferred embodiment, the composition of the invention is an ointment, LM030 is present in an amount of about 1% (w/w) of the composition, in solubilized form with diethylene glycol monoethyl ether is the solubilizing agent, which is present in an amount of about 5% to about 10% (w/w) of the composition; the hydrophobic matrix comprises a combination of petrolatum, mineral oil and microcrystalline wax; and isopropyl myristate is incorporate as a consistency enhancer in an amount of about 5% (w/w).

In a further embodiment of the invention, the pharmaceutical compositions of LM030 described herein are administered topically to a patient in need thereof for the treatment of Netherton's Disease. The topical pharmaceutical compositions will comprise an effective amount of LM030, preferably in an amount of from about 0.1% to about 5% (w/w), most preferably in an amount of about 0.5% to about 1% (w/w). The compositions may be applied on affected area on the body either once a day or, preferably, twice a day or on as-needed bases more than twice a day. The composition may be used chronically or on an as-needed bases.

Methods of making formulations of the present invention entail solubilizing the active agent, LM030, with the solubilizing agent, and combining the resulting solution with excipients comprising the quantities desired for the final formulation. In some embodiments, the active agent will be solubilized with the solubilizing agent alone, before combination with additional excipients. In other embodiments, the active agent will be solubilized with the solubilizing agent and other additional excipients in a single step. Additional excipients, including a consistency enhancer as well as other excipients, may be included to obtain the consistency desired for the final formulation. Methods of preparing gel, lotion, ointment, cream or foam final dosage forms are known in the art.

In one embodiment, the dosage form is an ointment. In this embodiment, the hydrophobic matrix materials, with or without a consistency enhancer, are heated to about 70° C. to 80° C. and stirred to obtain a melt, then cooled to about 45° C. to 55° C. with stirring. The solution containing LM030 and the solubilizing agent is added while stirring, then homogenized. The composition is then cooled to about 30° C. to 40° C., and loaded into the final container (e.g., tube). Preferably, the active agent is solubilized with diethylene glycol monoethyl ether (Transcutol HP), and the hydrophic matrix comprises a mixture of mineral oil, white petrolatum, white bees wax (microcrystalline wax), and isopropyl myristate.

In another embodiment, the dosage form is a gel. In this embodiment, the LM030 is solubilized by mixing with the solubilizing agents and other excipients such as antioxidants and preservatives at room temperature until clear. In a separate vessel, matrix materials are thoroughly mixed at room temperature, then a gelling excipient is added and mixed at room temperature until a clear gel forms. The solubilized LM030 mixture is added to the gel mixture and mixed at room temperature until a clear gel is obtained.

The invention is further illustrated by the following Examples.

### EXAMPLES

### Example 1

A prior art ointment suspension formulation described in U.S. Pat. No. 8,680,054 was prepared by the method described therein, having the composition described in Table 1 below.

TABLE 1
Formula A
Quantitative and qualitative composition of LM030 1% w/w
original ointment suspension formulation

| | Amount | | Reference |
|---|---|---|---|
| Ingredient | (mg) | Function | Standards |
| LM030 | 10.00 | Active | |
| | | ingredient | |
| White Petrolatum | 535.00 | Matrix | USP |
| Paraffin, Liquid/Mineral Oil | 300.00 | Matrix | Ph Eur/USP |
| Microcrystalline Wax | 125.00 | Matrix | NF |
| Isopropyl Myristate | 30.00 | Matrix | Ph Eur/NF |
| Total | 1000.00 | | |

NF: National formulary,
Ph Eur: European Pharmacopoeia,
USP: United States Pharmacopeia

In addition, ointment formulations containing LM030 solubilized with a solubilizing agent were prepared by combining the excipients described in Examples 2-5 below.

### Example 2

TABLE 2
Formula B
Quantitative and qualitative composition of LM030 1% w/w
formulation containing 10% Transcutol HP

| | Amount | | Reference |
|---|---|---|---|
| Ingredient | (mg) | Function | Standards |
| LM030 | 10.00 | Active | |
| | | ingredient | |
| White Petrolatum | 620.00 | Matrix | USP |
| Paraffin, Liquid/Mineral Oil | 200.00 | Matrix | Ph Eur/USP |
| Microcrystalline Wax | 20.00 | Matrix | NF |
| Isopropyl Myristate | 50.00 | Matrix | Ph Eur/NF |
| Transcutol HP | 100.00 | Solubilizing | NF |
| | | agent | |
| Total | 1000.00 | | |

NF: National formulary,
Ph Eur: European Pharmacopoeia,
USP: United States Pharmacopeia

Formula B was prepared by combining LM030 with Transcutol HP at room temperature and mixing at 500-700 rpm to obtain a clear solution. Separately, the white petrolatum, mineral oil, microcrystalline wax and isopropyl myristate were combined in a vessel and heated to 70° C. to 80° C. with mixing at 100 to 200 rpm to obtain a melt. The melt was cooled to 45° C. to 55° C., with stirring at 100 to 300 rpm and the solution containing LM030 was added with stirring. The combination was homogenized at 5500 rpm for 3 minutes. The composition was cooled to 30° C. to 40° C. with stirring at 100 to 300 rpm, and the resulting composition was loaded into 30 g aluminum tubes.

### Example 3

Formula C was prepared by the same process as Formula B, with the composition shown in Table 3.

TABLE 3
Formula C
Quantitative and qualitative composition of LM030 1% w/w
formulation containing 2.5% Transcutol HP and 2.5% Oleic acid
LM030 Topical Ointment, 1%
With 2.5% Transcutol and 2.5% Oleic Acid

| Material | Grade | % w/w |
|---|---|---|
| White Petrolatum | USP | 67.00 |
| Mineral Oil | USP | 20.00 |
| White Bees Wax | NF | 2.00 |
| Isopropyl Myristate | NF | 5.00 |
| Transcutol HP | NF | 2.50 |
| Oleic Acid | NF | 2.50 |
| LM030 | IH | 1.00 |

| | |
|---|---|
| Total | 100.00 |

### Example 4

Formula D was prepared by the same process as Formula B, with the composition shown in Table 4.

TABLE 4
Formula D
Quantitative and qualitative composition of LM030
1% w/w formulation containing 2.5% Transcutol
LM030 Topical Ointment, 1%
With 2.5% Transcutol

| Material | Grade | % w/w |
|---|---|---|
| White Petrolatum | USP | 69.50 |
| Mineral Oil | USP | 20.00 |
| White Bees Wax | NF | 2.00 |
| Isopropyl Myristate | NF | 5.00 |
| Transcutol HP | NF | 2.50 |
| LM030 | IH | 1.00 |

| | |
|---|---|
| Total | 100.00 |

Example 5

Formula E was prepared by the same process as Formula B, with the composition shown in Table 5.

TABLE 5
Formula E
Quantitative and qualitative composition of LM030
1% w/w formulation containing 5% Transcutol
LM030 Topical Ointment, 1%
With 5% Transcutol

| Material | Grade | % w/w |
|---|---|---|
| White Petrolatum | USP | 67.00 |
| Mineral Oil | USP | 20.00 |
| White Bees Wax | NF | 2.00 |
| Isopropyl Myristate | NF | 5.00 |
| Transcutol HP | NF | 5.00 |
| LM030 | IH | 1.00 |

| | |
|---|---|
| Total | 100.00 |

### Example 6

A topical gel formulation containing 1% LM030 was prepared with the composition shown in Table 6, according the following process. Propylene glycol, methylparaben and propylparaben were added to a vessel and mixed together at room temperature using a mixer speed of 200-400 rpm until a clear solution was obtained. Then hydroxypropyl cellulose was added and mixed at room temperature and mixer speed of 300-600 rpm until a clear gel was obtained (Portion A). In another vessel, LM030 was mixed with the following excipients: diethylene glycol monoethyl ether (Transcutol HP), Isopropyl myristate, citric acid monohydrate, and sodium phosphate dibasic hydrate. They were mixed at room temperature and mixing speed of 500-700 rpm until clear (Portion B). Portion B then was added to Portion A and mixed at room temperature for 60 minutes at mixing speed of 500-700 rpm until a clear gel was obtained.

TABLE 6
Formula F
Quantitative and qualitative composition of LM030
1% w/w gel formulation containing 10% Transcutol

| MATERIAL | GRADE | % w/w |
|---|---|---|
| Propylene glycol | USP | 77.57 |
| Transcutol HP | NF | 10.00 |
| Isopropyl Myristate | NF | 10.00 |
| Hydroxypropylcellulose (Klucel HF) | NF | 1.20 |
| Citric acid monohydrate | USP | 0.005 |
| Sodium phosphate dibasic dihydrate | USP | 0.005 |
| Methylparaben | USP | 0.20 |
| Propylparaben | USP | 0.02 |
| LM030 | IH | 1.00 |

| | |
|---|---|
| Total | 100.00 |

### Example 7

### Stability Tests

The following table shows the stability of LM030 when formulated as a suspension formulation and when formulated in solubilized form, under the same conditions of temperature and time.

TABLE 7

| | % degradation |
|---|---|
| Formulations of LM030 stored at 50° C. for 7 days | of LM030 |
| LM030 solubilized in water | 12% |
| LM030 solubilized in water and 0.1% ascorbic acid | 11% |
| LM030 solubilized in phosphate buffer stored at pH 5 | 15% |
| (at 50 C., 1 Week) | |
| LM030 solubilized in phosphate buffer at pH 8 | 8% |
| (at 50 C., 1 Week) | |
| LM030 solubilized in PEG300 | 5.4% |
| LM030 solubilized in oleylalchol | stable |
| LM030 solubilized in propylene carbonate | stable |
| LM030 solubilized in DMI | 4.5 |
| LM030 solubilized in Transcutol HP | stable |
| LM030 1% w/w suspension (original Formula A) | stable |
| LM030 solubilized in glycerol/propylene glycol 1:1 | 2.9% |
| LM030 solubilized in propylene glycol | 0.9% |
| LM030 suspended in isoproylmyristate/propylene glycol 1:1 | Stable |
| LM030 in 0.1% ascorbic acid/water at 50 C., 1 week | 11% |

Based on data presented above, LM030 showed poor stability in the presence of aqueous solutions, but showed acceptable stability when solubilized in certain solubilizers. Most solvents that showed good stability, however, also showed limited solubility, thereby providing insufficient exposure for producing the desired therapeutic effect.

The ointment Formula A (containing LM030 in suspension formulation) and Formulation B (containing LM030 solubilized in Transcutol HP) described in Examples 1 and 2 were tested for stability after storage for one or two weeks at room temperature and at 40° C. The results are shown in Table 8.

TABLE 8

| Time | | Formula A | | Formula B | |
|---|---|---|---|---|---|
| (weeks) | Parameter | 25° C. | 40° C. | 25° C. | 40° C. |
| 0 | % Potency | 107.3 | 107.3 | 101.2 | 101.2 |
| | Viscosity | 1692 | 1692 | 260.6 | 260.6 |
| | % Single largest | 0.96 | 0.96 | 1.236 | 1.26 |
| | Impurity RRT = | | | | |
| | 0.83 | | | | |
| | Total Related | 1.1 | 1.1 | 1.38 | 1.38 |
| | Impurity | | | | |
| 1 | % Potency | 102.9 | 102.6 | 102.8 | 113.5/104.6 |
| | Viscosity | 1673 | 1684 | 260.2 | 265.1 |
| | % Single largest | 1.14 | 1.15 | 0.42 | 1.24 |
| | Impurity RRT 0.83 | | | | |
| | Total Related | 1.38 | 1.28 | 1.57 | 1.78 |
| | Impurity | | | | |
| 2 | % Potency | 104.8 | 98.3 | 102.0 | 98.9/102.6 |
| | Viscosity | 1599 | 1733 | 168.4 | 259.2 |
| | % Single largest | 1.24 | 1.5 | 0.55 | 1.00 |
| | Impurity RRT 0.83 | | | | |
| | Total Related | 1.67 | 1.69 | 0.55 | 1.30 |
| | Impurity | | | | |

Formula B showed comparable stability under both standard and accelerated conditions, as compared to prior art Formula A. LM030 was solubilized in Formulation B while it was in a suspension form in Formulation A.

In addition, Formula C was compared to the prior art Formula A of Example 1, and tested for stability after storage for one or two weeks at room temperature and at 40° C. The results are shown in Table 9.

TABLE 9

| Time | | Formula A | | Formula C | |
|---|---|---|---|---|---|
| (weeks) | Parameter | 25° C. | 40° C. | 25° C. | 40° C. |
| 0 | % Potency | 101.27 | 101.27 | 91.7 | 91.7 |
| | Viscosity | 1910 | 1910 | 333.1 | 333.1 |
| | % Single largest | 1.30 | 1.30 | 1.61 | 1.61 |
| | Impurity RRT = | | | | |
| | 0.83 | | | | |
| | Total Related | 1.44 | 1.44 | 1.79 | 1.79 |
| | Impurity | | | | |
| 1 | % Potency | 100.6 | 102.1 | 110.4 | 100.9 |
| | Viscosity | 1892 | 1925 | 325.4 | 325.4 |
| | % Single largest | 1.30 | 1.31 | 1.51 | 0.85 |
| | Impurity RRT 0.83 | | | | |
| | Total Related | 1.42 | 1.48 | 1.68 | 1.61 |
| | Impurity | | | | |
| 2 | % Potency | 102.1 | 103.3 | 104.6 | 118.8 |
| | Viscosity | 1765 | 1800 | 300.1 | 317.1 |
| | % Single largest | 1.40 | 1.38 | 1.14 | 1.29 |
| | Impurity RRT 0.83 | | | | |
| | Total Related | 1.56 | 1.64 | 1.30 | 1.87 |
| | Impurity | | | | |

Formula C showed comparable stability to Formula A under both conditions.

### Example 8

### Pharmacokinetic Studies

Pharmacokinetic studies were performed using young, female Gottingen minipigs. Test formulations were applied to three animals per group, BID for two days, with application of 15% surface area. Samples were obtained from plasma, as well as the dermis and epidermis. For separation of epidermis and dermis, following tape stripping the punch biopsies were obtained. The veterinarian then used a scalpel to separate the two layers with the aid of a dissecting microscope. The two layers were then placed in tubes and frozen until analysis.

TABLE 10
PK Results

| | | Plasma | | | | | | | |
|---|---|---|---|---|---|---|---|---|---|
| | | Concentration | | | | | | | |
| | | 1 hr | | | | | | | |
| | | post- | | | | | | | |
| | | 2nd | 12 hr | | | | | | |
| | | admin. | post- | Day 2 am Epidermis (ng/g) | | | Day 2 am Dermis (ng/g) | | |
| Treatment | | Mean | 2nd | | Mean | | | Mean | |
| Group | Animal | (SD) | admin. | Individual | (SD) | Median | Individual | (SD) | Median |
| Formula A | 1823 | 2.2 | <0.5 | 86,590 | 247,190 | 91,690 | 1,158 | 5,400 | 3,903 |
| (suspension | 1833 | (2.2) | | 563,280 | (273,754) | | 11,140 | (5,157) | |
| formulation) | 1840 | | | 91,690 | | | 3,903 | | |
| Formula E | 1853 | 1.0 | <0.5 | 148,370 | 302,130 | 242,150 | 5,610 | 8,554 | 5,610 |
| (LM030 | 1841 | (0.8) | | 242,150 | (190,939) | | 5,203 | (5,456) | |
| solubilized | 1837 | | | 515,880 | | | 14,850 | | |
| in 5% | | | | | | | | | |
| Transcutol | | | | | | | | | |
| HP) | | | | | | | | | |
| Formula C | 1845 | 1.8 | <0.5 | 206,830 | 183,430 | 206,830 | 11,197 | 7,325 | 6,110 |
| (LM030 | 1842 | (1.0) | | 106,520 | (68,244) | | 6,110 | (3,430) | |
| solubilized | 1829 | | | 237,060 | | | 4,667 | | |
| in 2.5% | | | | | | | | | |
| Transcutol | | | | | | | | | |
| HP, 2.5% | | | | | | | | | |
| oleic acid) | | | | | | | | | |

In a second PK study, comparing ointment Formulas A and B from Example 1, and the gel formulation from Example 6 (Formula F), test formulations were compared in minipigs as described above, with administration twice daily for seven days. The results are shown in Table 11. No adverse findings, nor notable differences between groups were found in food consumption, body weight gain, clinical observations, and Draize scoring (erythema, edema).

TABLE 11
PK Results

| | | | Plasma cone. | | Dermis conc. \hat{ } ng/g | | Epidermis conc. \hat{ } ng/g | |
|---|---|---|---|---|---|---|---|---|
| | | | ^{#} ng/ml | | Mean (SD) | | Mean (SD) | |
| Group | Formulation | N | Day 1 | Day 7 | Day 1 | Day 7 | Day 1 | Day 7 |
| 1 | Formula A | 3 | ≤0.5* | ≤0.5 | 1350 (567) | 7880 (4679) | 33228 | 89693 (65858) |
| | | | | | | | (18699) | |
| 2 | Formula B | 3 | ≤0.5* | ≤0.5* | 2742 (993) | 16983 | 53883 | 211558 |
| | | | | | | (13392) | (19816) | (43410) |
| 3 | Formula F | 3 | ≤0.5 | ≤0.5 | 374 (441) | 990 (370) | 4723 (4280) | 14144 (3387) |

*1-2 animals per group with 0.5-5 ng/ml levels. LLOQ 0.5 ng/ml plasma, 5 ng/g tissue
^{#} peak levels any time point
\hat{ } approximately 7.5 hr after am application

The test formulations containing 5% Trancutol, 10% Transcutol and 2.5% Transcutol/2.5% oleic acid as solubilizing agents all showed improved LM030 absorption in the dermis and epidermis, as compared to the original suspension formulation, while also maintaining low systemic exposure and no notable increase in adverse events.

### Example 9

### Stability Data

A formulation of the invention containing the following composition was prepared.

| | | % LM-030 1% |
|---|---|---|
| Material | Grade* | ointment |
| White Petrolatum | USP | 67.00 |
| Mineral Oil | USP | 20.00 |
| White Bees Wax | NF | 2.00 |
| Iso propyl myristate | NF | 5.00 |
| Transcutol HP | NF | 5.00 |
| LM-030 | IH | 1.00 |
| | | 100% |

Stability was tested at three months, six months and nine months under the following three conditions: 5° C., 25° C./60%RH, and 30° C./65%RH. The formulation showed good stability under all conditions, as shown in Tables 12, 13 and 14.

TABLE 12
Stability Data at 5° C.

| | | 0 Month | | | 3 Month | 6 Month | 9 Month | |
|---|---|---|---|---|---|---|---|---|
| Test | Specification | Beg | Mid | End | Mid | Mid | Mid | End |
| Physical | Translucent | Conform | Conform | Conform | Conform | Conform | Conform | |
| Appearance | Homogeneous | | | | | | | |
| | ointment | | | | | | | |
| Appearance | White aluminum tubes | Conform | Conform | Conform | Conform | Conform | Conform | |
| of | with internal protective | | | | | | | |
| Container | lacquer, without imprint, | | | | | | | |
| | with membrane. White | | | | | | | |
| | screw cap with a built- in | | | | | | | |
| | point | | | | | | | |
| Identity by | Positive | Conform | Conform | Conform | Conform | Conform | Conform | |
| HPLC | | | | | | | | |
| Viscosity | Report results | 292.5 cPs | 246.4 cPs | 263.4 cPs | 313.9 cPs | 334.0 cPs | 330.3 cPs | |
| Tube | Uniform translucent | Conform | Conform | Conform | Conform | Conform | Conform | |
| uniformity | homogeneous ointment, | | | | | | | |
| | with no phase separation | | | | | | | |
| Tube | Individual and mean % | TOP 98.5% | TOP 983% | TOP 98.6% | TOP 104.9% | TOP 104.5% | TOP 105 8% | TOP 104.5% |
| uniformity | assay should be within | MID 97.6% | MID 97.9% | MID 98.0% | MID 104.2% | MID 105.1% | MID 103.9% | MID 104.6% |
| | 90.0-110.0% of LC. | Bottom | Bottom 98.0% | Bottom 98.3% | Bottom 104.1 % | Bottom 104.3% | Bottom | Bottom |
| | maximum difference | 98.5% | Max. Diff 0.4 | Max. Diff 0.6 | Max. Diff 0.8 | Max. Diff 0.8 | 104.5% Max. | 104.2% Max. |
| | in the amount of active | Max. | | | | | Diff 1.9 | Diff 0.3 |
| | ingredient determined | Diff 0.9 | | | | | | |
| | within the tube is NMT | | | | | | | |
| | 10.0% | | | | | | | |
| Assay for | 90-110.0% | 98.2%* | 98.1%* | 98.3%* | 104.4% | 104.7% | 104.7% | 104.5% |
| LM030 | | | | | | | | |
| Total | Report results | 1.36% | 1.36% | 1.54% | 1.62% | 1.52% | 0.82% | 0.78% |
| Impurities | | | | | | | | |

*Lower Assay results at T = 0M was due to sample solution

TABLE 13
Stability Data at 25° C./60% RH

| | | 0 Month | | | 3 Month | 6 Month | 9 Month |
|---|---|---|---|---|---|---|---|
| Test | Specification | Beg | Mid | End | Mid | Mid | Mid |
| Physical | Translucent Homogeneous | Conform | Conform | Conform | Conform | Conform | Conform |
| Appearance | ointment | | | | | | |
| Appearance | White aluminum tubes | Conform | Conform | Conform | Conform | Conform | Conform |
| of Container | with internal protective | | | | | | |
| | lacquer, without imprint, | | | | | | |
| | with membrane. White | | | | | | |
| | screw cap with a built-in | | | | | | |
| | point | | | | | | |
| Identity by | Positive | Conform | Conform | Conform | Conform | Conform | Conform |
| HPLC | | | | | | | |
| Viscosity | 350 +/− 150 cPs | 292.5 cPs | 246.4 cPs | 263.4 cPs | 290.7 cPs | 315.7 cPs | 314.6 cPs |
| Tube | Uniform translucent | Conform | Conform | Conform | Conform | Conform | Conform |
| uniformity | homogeneous ointment, | | | | | | |
| | with no phase separation | | | | | | |
| Tube | Individual and mean % | TOP 98.5% | TOP 98.3% | TOP 98.6% | TOP 103.2% | TOP 104.7% | TOP 103.7% |
| uniformity | assay should be within 90.0- | MID 97.6% | MID 97.9% | MID 98.0% | MID 103.3% | MID 102.6% | MID 104.1% |
| | 110.0% of LC. maximum | Bottom 98.5% | Bottom 98.0% | Bottom 98.3% | Bottom 102.9% | Bottom 105.7% | Bottom 103.9% |
| | difference in the amount of | Max. Diff 0.9 | Max. Diff 0.4 | Max. Diff 0.6 | Max. Diff 0.4 | Max. Diff 3.1 | Max. Diff 0.3 |
| | active ingredient determined | | | | | | |
| | within the tube is NMT | | | | | | |
| | 10.0% | | | | | | |
| Assay for | 90-110.0% | 98.2%* | 98.1%* | 98.3%* | 103.2% | 104.3% | 103.9% |
| LM030 | | | | | | | |
| Total | Report results | 1.36% | 1.36% | 1.54% | 1.67% | 1.56% | 1.68% |
| Impurities | | | | | | | |

*Lower Assay results at T = 0M was due to sample solution

TABLE 14
Stability Data at 30° C./65% RH

| | | 3 Month | 6 Month | 9 Month |
|---|---|---|---|---|
| Test | Specification | Mid | Mid | Mid |
| Physical | Translucent Homogeneous ointment | Conform | Conform | Conform |
| Appearance | | | | |
| Appearance | White aluminum tubes with internal protective lacquer, | Conform | Conform | Conform |
| of Container | without imprint, with membrane. White screw cap with a | | | |
| | built- in point | | | |
| Identity by | Positive | Conform | Conform | Conform |
| HPLC | | | | |
| Viscosity | 350 +/− 150 cPs | 307.6 cPs | 317.3 cPs | 311.0 cPs |
| Tube | Uniform translucent homogeneous ointment, with no | Conform | Conform | Conform |
| uniformity | phase separation | | | |
| Tube | Individual and mean % assay should be within 90.0-110.0% | TOP 103.0% | TOP 104.4% | TOP 103.9% |
| uniformity | of LC. maximum difference in the amount of active | MID 103.8% | MID 104.1% | MID 103.8% |
| | ingredient determined within the tube is NMT 10.0% | Bottom 106.0% | Bottom 105.5% | Bottom 102.1% |
| | | Max. Diff 3.0 | Max. Diff 1.4 | Max. Diff 1.9 |
| Assay for | 90-110.0% | 104.3% | 104.7% | 103.3% |
| LM030 | | | | |
| Total | Report results | 1.66% | 1.62% | 2.00% |
| Impurities | | | | |

* * *

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A pharmaceutical composition comprising the compound of Formula I 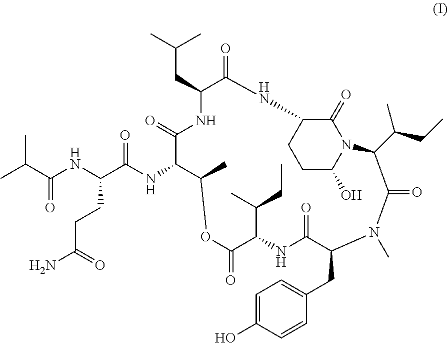 [SMILES: CC[C@H](C)[C@@H]1NC(=O)[C@H](Cc2ccc(O)cc2)N(C)C(=O)[C@H]([C@@H](C)CC)N2C(=O)[C@H](CC[C@H]2O)NC(=O)[C@H](CC(C)C)NC(=O)[C@@H](NC(=O)[C@H](CCC(N)=O)NC(=O)C(C)C)[C@@H](C)OC1=O]
in solubilized form with a solubilizing agent,
and one or more pharmaceutically acceptable excipients in a formulation suitable for topical administration.

2. The pharmaceutical composition of claim 1 wherein the solubilizing agent is selected from the group consisting of diethylene glycol monoethyl ether, medium chain triglycerides, fatty acids, propylene glycol and combinations thereof.

3. The pharmaceutical composition of claim 2 wherein the solubilizing agent comprises diethylene glycol monoethyl ether.

4. The pharmaceutical composition of claim 2 wherein the solubilizing agent comprises a mixture of diethylene glycol monoethyl ether and a fatty acid.

5. The pharmaceutical composition of claim 2 wherein the solubilizing agent comprises oleic acid.

6. The pharmaceutical composition of claim 2 wherein the solubilizing agent is a medium chain triglyceride or a mixture of medium chain triglycerides.

7. The pharmaceutical composition of claim 8 wherein the solubilizing agent is Labrofac® lipophile WL 1349.

8. The pharmaceutical composition of claim 8 wherein the solubilizing agent is Miglyol® triglycerides.

9. The pharmaceutical composition of claim 1 wherein the composition is a gel, an ointment, a cream, a lotion, a spray, or a foam.

10. The pharmaceutical composition of claim 1 wherein the solubilized compound of Formula 1 is within a hydrophobic matrix comprising one or more excipients selected from the group consisting of paraffins, vegetable oils, animal fats, synthetic glycerides, waxes, perfluorocarbons, semiperfluorocarbons, and liquid polysiloxanes.

11. The pharmaceutical composition of claim 1 wherein the compound of formula I is present in an amount of from about 0.1 to about 5% w/w of the composition.

12. The pharmaceutical composition of claim 11 wherein the solubilizing agent is present in an amount of from about 5% to about 25% (w/w) of the composition.

13. The pharmaceutical composition of claim 12 wherein the solubilizing agent comprises diethylene glycol monoethyl ether in an amount of about 10% (w/w) of the composition.

14. The pharmaceutical composition of claim 12 wherein the solubilizing agent comprises diethylene glycol monoethyl ether in an amount of about 5% (w/w) of the composition.

15. The pharmaceutical composition of claim 12 wherein the solubilizing agent comprises a mixture of diethylene glycol monoethyl ether and oleic acid.

16. The pharmaceutical composition of claim 15 wherein the diethylene glycol monoethyl ether is in an amount of about 2.5% (w/w) of the composition and the oleic acid is in an amount of about 2.5% (w/w) of the composition.

17. A pharmaceutical composition suitable for topical administration comprising:
a compound of Formula I 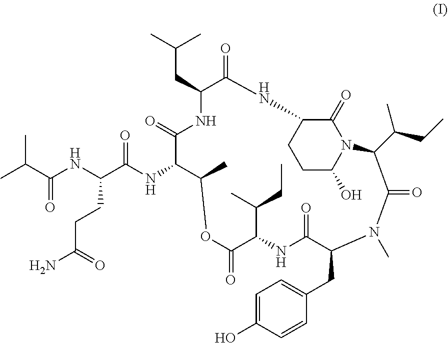 [SMILES: CC[C@H](C)[C@@H]1NC(=O)[C@H](Cc2ccc(O)cc2)N(C)C(=O)[C@H]([C@@H](C)CC)N2C(=O)[C@H](CC[C@H]2O)NC(=O)[C@H](CC(C)C)NC(=O)[C@@H](NC(=O)[C@H](CCC(N)=O)NC(=O)C(C)C)[C@@H](C)OC1=O]
in solubilized form with diethylene glycol monoethyl ether as a solubilizing agent; and
a hydrophobic matrix comprising one or more excipients selected from the group consisting of petrolatum, mineral oil, microcrystalline wax and combinations thereof.

18. The pharmaceutical composition of claim 17 further comprising isopropyl myristate as a consistency enhancer.

19. A method for treating Netherton's disease comprising administering the pharmaceutical composition of claim 1.

20. A method of treating atopic dermatitis comprising administering the pharmaceutical composition of claim 1.
